(19) United States
(12) Patent Application Publication
(10) Pub. No.: US 2006/0235242 A1
(43) Pub. Date: Oct. 19, 2006

(54) Recovering method of acetic acid from effluent of terephthalic acid production process

(75) Inventor: Ki Joon Kang, Gyeonggi-do (KR)

Correspondence Address: AKERMAN SENTERFITT, 801 PENNSYLVANIA AVENUE N.W., SUITE 600, WASHINGTON, DC, 20004, US

(73) Assignees: Ki Joon KANG, Gyeonggi-do (KR); AMTPACIFIC CO., LTD., Seoul (KR)

(21) Appl. No.: 11/403,928

(22) Filed: Apr. 14, 2006

(30) Foreign Application Priority Data
Apr. 14, 2005 (KR) 10-2005-0030921
Mar. 22, 2006 (KR) 10-2006-0026226

(51) Int. Cl. C07C 51/16; C07C 51/42

(52) U.S. Cl. 562/410; 562/608

(57) ABSTRACT

The present invention relates to a recovering method of acetic acid using an apparatus comprising a dehydration tower, a condenser and a separation tank. As p-xylene is used as an azeotropic agent to form an azeotrope with water, which is discharged from the top of the dehydration tower, and the remaining p-xylene is discharged from the bottom of the dehydration tower along with acetic acid, the acetic acid containing the p-xylene azeotropic agent can be recycled directly to a reactor for producing terephthalic acid without further separation process and without regard to the content of the p-xylene azeotropic agent which is recovered along with acetic acid.

### CROSS-REFERENCE TO RELATED APPLICATIONS

The present application claims priority from Korean Patent Applications Nos. 10-2005-0030921, filed Apr. 14, 2005, and 10-2006-0026226, filed Mar. 22, 2006, the contents of which are herein incorporated by reference in their entirety.

### TECHNICAL FIELD

The present invention relates to a recovering method of acetic acid from the effluent of terephthalic acid production processes using p-xylene as an azeotropic agent. When water containing acetic acid and p-xylene are supplied to the azeotropic distillation tower, water and p-xylene is discharged from the top of the column and dehydrated acetic acid is recovered from the bottom of the column. Since p-xylene is a reactant of the terephthalic acid production reaction, further separation of p-xylene from the recovered acetic acid is not necessary even if p-xylene is contained in the acetic acid.

### BACKGROUND ART

In general, terephthalic acid is obtained from the catalytic reaction of p-xylene with oxygen in the air using acetic acid as a solvent. In the reaction, water is produced along with terephthalic acid. Since the cost of acetic acid recovery takes a high proportion in the production cost of terephthalic acid, a lot of researches are being performed to effectively remove water from acetic acid.

Commonly known methods for removing water from acetic acid are conventional distillation, azeotropic distillation, extraction, adsorption, and so forth.

FIG. 1 illustrates a process of recovering acetic acid by conventional distillation.

Referring to FIG. 1, an apparatus for recovering acetic acid by conventional distillation comprises a dehydration tower (1) for separating acetic acid and water by distillation, a condenser (2) for condensing the vapor discharged from the top of the dehydration tower (1), a collection tank (3) for collecting liquid water and acetic acid that have passed through the condenser (2) and a heater (4) for supplying energy to the dehydration tower (1). Reaction product, or a mixture of water and acetic acid, of a terephthalic acid production process is supplied to the dehydration tower via a pipe (L1).

Under normal pressure, acetic acid boils at about 118° C. and water boils at about 100° C. In spite of the boiling point difference of about 18° C., reflux ratio at the top of the dehydration tower (1) has to be maintained at 3-6 or above for a mixture having a low acetic acid concentration in order to eliminate water from the top of the dehydration tower (1), because diluted acetic acid aqueous solutions are close boiling mixtures. Since the reflux ratio is high in a conventional distillation process, energy consumption in the process becomes 4-7 times of the heat of vaporization of water.

Despite such large energy consumption, the wastewater discharged by a pipe (L2) contains 0.3-0.8 wt % of acetic acid. That is, the conventional distillation is ineffective in acetic acid recovery and requires additional cost for the wastewater treatment.

Several techniques utilizing azeotropic distillation have been proposed to reduce energy consumption at the dehydration tower and maximize recovery of acetic acid contained in water. Korean Patent No. 45509 is an example.

In this patent, an azeotropic agent is added to a mixture of water and acetic acid to obtain an azeotrope. Because the resultant azeotrope boils at a temperature lower than the boiling temperature of water, energy consumption at the dehydration tower can be reduced to about 60-70% compared to that in a conventional distillation. For the azeotropic agent, isobutyl acetate, n-butyl acetate, etc. are used.

However, these azeotropic agents have some shortcomings. When a small amount of the azeotropic agent is discharged with acetic acid from the bottom of the dehydration tower and it is introduced to the p-xylene oxidation reactor, the azeotropic agent can be destroyed. In order to solve this problem, a lot of energy is required to prevent the inflow of the azeotropic agent into the dehydrated acetic acid in the dehydration tower. In case of an accident in which large amount of the azeotropic agent is poured into the reboiler of the dehydration tower, it is not easy to separate the azeotropic agent from acetic acid in the bottom of the tower. Thus, it has to be discharged from the tower or stored in a tank and small amount of the mixture should be supplied to the dehydration tower with the main feed to recover the azeotropic agent. This requires additional cost.

When n-butyl acetate is used as an azeotropic agent, the azeotropic temperature is about 90° C. When unreacted p-xylene is introduced to the dehydration tower, p-xylene and water mixture also forms an azeotrope at about 94° C. Since the boiling temperature of p-xylene and water azeotrope is higher than that of n-butyl acetate and water azeotrope and it is lower than that of acetic acid, p-xylene is neither discharged from the top of the dehydration tower nor discharged from the bottom of the tower. As a result, p-xylene may be accumulated inside the dehydration tower. In order to prevent this phenomenon, p-xylene separation tower has to be equipped.

### DISCLOSURE OF THE INVENTION

An object of the present invention is to provide a recovering method of acetic acid from effluent from a terephthalic acid production process capable of preventing accumulation of p-xylene inside the dehydration tower, which may occur when isobutyl acetate or n-butyl acetate is used as an azeotropic agent, and thereby saving energy required for recovering acetic acid.

Another object of the present invention is to provide a recovering method of acetic acid from effluent in a terephthalic acid production process capable of preventing corrosion at the bottom of the dehydration tower, caused by high-concentrated acetic acid, by maintaining high concentration of p-xylene in the bottom of the tower.

To attain the objects, the present invention provides a recovering method of acetic acid from effluent from a terephthalic acid production process using an apparatus comprising:
a dehydration tower for recovering acetic acid from a mixture of water and acetic acid, which is produced from the catalytic oxidation of p-xylene using acetic acid as a solvent, by azeotropic distillation using an azeotropic agent;
a condenser for condensing gaseous azeotropic agent and water discharged from the top of the dehydration tower; and
a separation tank for removing water by separating the azeotropic agent from water,
wherein p-xylene is used as the azeotropic agent to form an azeotrope with water, which is discharged at the top of the dehydration tower, and remaining p-xylene is discharged at the bottom of the dehydration tower along with acetic acid.

p-Xylene, which is used as the azeotropic agent in the present invention, is supplied from the separation tank and is preferably comprised in the gaseous azeotrope discharged at the top of the dehydration tower within 1.95-2.2 parts by weight per 1 part by weight of water.

If the supply of p-xylene is less than the proper amount specified above, part of the water removed at the top of the dehydration tower is removed without forming an azeotrope, which causes additional energy consumption in the tower. In contrast, if the supply of p-xylene is larger than the proper amount, excess p-xylene is discharged from the bottom of the dehydration tower along with acetic acid. Consequently, the content of p-xylene in the gaseous azeotrope discharged at the top of the dehydration tower cannot exceed 2.2 parts by weight per 1 part by weight of water. If excess p-xylene is forced to be discharged from the top of the dehydration tower, a large amount of acetic acid may be included in the gas phase and the energy consumption increases.

The p-xylene containing acetic acid, which is discharged from the bottom of the dehydration tower, is recycled into the reactor for producing terephthalic acid without an additional process for separating the p-xylene azeotropic agent. The content of p-xylene included in the acetic acid can be 0-65 parts by weight, preferably 0-30 parts by weight, per 100 parts by weight of the total effluent discharged from the bottom of the dehydration tower. A p-xylene content exceeding 65 parts by weight is undesirable in view of operation load of the dehydration tower because excessive p-xylene is supplied to the reactor.

In the separation tank where p-xylene is separated from water, part of water in the lower layer is refluxed to the dehydration tower and the remainder is discharged as wastewater. p-Xylene included in the upper layer is refluxed to the dehydration tower. Supply of p-xylene is required to compensate for the amount discharged from the bottom of the dehydration tower.

If the pressure at the top of the dehydration tower is higher than normal pressure, temperature inside the condenser increases compared with operation at normal pressure. Consequently, the condenser may act as a steam generator and contribute to energy recovery.

In addition top-xylene, the azeotropic agent may further comprise: methyl acetate, which is produced as byproduct in a terephthalic acid production process; isobutyl acetate or n-butyl acetate, which remain when the azeotropic agent is changed from isobutyl acetate or n-butyl acetate to p-xylene; or butanol, which is produced as byproduct when butyl acetate is used as azeotropic agent. Preferably, p-xylene is comprised within 50-100 wt % per 100 wt % of the total azeotropic agent.

If the content of p-xylene in the total azeotropic agent is below 50 wt %, azeotropic agent components other than p-xylene may be discharged from the bottom of the tower and loss of the azeotropic agent included in water in the separation tank (12) tends to increase. Thus, it is preferable to maintain the content of p-xylene high, for example at 100 wt %.

### BRIEF DESCRIPTION OF THE DRAWINGS

FIG. 1 illustrates a recovering method of acetic acid by conventional distillation.

FIG. 2 illustrates a recovering method of acetic acid from the effluent of a terephthalic acid production process according to the present invention.

### BEST MODE FOR CARRYING OUT THE INVENTION

Hereinafter, the present invention is described in further detail referring to FIG. 2.

FIG. 2 illustrates a recovering method of acetic acid from effluent of a terephthalic acid production process according to the present invention. The apparatus for recovering acetic acid from the effluent of the terephthalic acid production process in accordance with the present invention comprises a dehydration tower (10) for recovering acetic acid from a mixture of water and acetic acid, which is supplied from the terephthalic acid production process, using an azeotropic agent by azeotropic distillation, a condenser (11) for condensing gaseous azeotropic agent and water discharged from the top of the dehydration tower (10), a separation tank (12) for separating the azeotropic agent and water which have passed through the condenser (11) and a heater (13) for providing steam to the dehydration tower (10).

p-Xylene is supplied to the dehydration tower (10) as the azeotropic agent to form an azeotrope with water, which is discharged from the top of the dehydration tower, and dehydrated acetic acid is recovered from the bottom of the dehydration tower. Amount of the p-xylene azeotropic agent refluxed to the dehydration tower via a pipe (L14) is determined such that the content of p-xylene included in the gaseous azeotrope discharged from the top of the tower via a pipe (L12) is 1.95-2.2 parts by weight per 1 part by weight of water. If more p-xylene is supplied as the azeotropic agent than the amount discharged at the top of the tower, excess p-xylene is discharged from the bottom of the dehydration tower along with acetic acid and is recycled to the reactor.

For example, suppose that, 100 g/hr of the source material supplied via a pipe (L11), 30 wt % is water and water refluxed via a pipe (L16) is 30 g/hr and water discharged via a pipe (L15) at the bottom of the dehydration tower is 5 g/hr. Then, water discharged via the pipe (L12) is 55 g/hr (30 g/hr +30 g/hr −5 g/hr). Accordingly, the amount of p-xylene at the pipe (L12) should be in the range from 107.25 g/hr (55×1.95) to 121 g/hr (55×2.2).

If the amount of p-xylene discharged from the bottom of the tower via the pipe (L15) is 1 g/hr, the amount of p-xylene refluxed via the pipe (L14) becomes 108.25-122 g/hr.

Amount of water discharged from the top of the tower via the pipe (L12) is determined by the amount of water supplied to the tower via the pipe (L11), the concentration of acetic acid discharged from the bottom of the tower via the pipe (L15) (about 90-95 wt %) and the amount of water refluxed via the pipe (L16). The pipe (L14) and the pipe (L16) may be installed at the top or middle of the tower and may be more than one.

Preferably, the content of p-xylene refluxed to the dehydration tower via the pipe (L14) after being separated at the separation tank (12) is 99 wt % or higher. However, in practice, a small amount of methyl acetate produced as byproduct may be included. Methyl acetate forms an azeotrope together with water. Since it forms an azeotrope at a concentration of 20 parts by weight or more per 1 part by weight of water, operation load of the dehydration tower may increase if the content of methyl acetate included in the p-xylene azeotropic agent refluxed to the dehydration tower via the pipe (L14) becomes larger. Accordingly, it is preferable to continuously discharge methyl acetate using an additional tower.

More specifically, a mixture of water and acetic acid, which is supplied from a reactor for producing terephthalic acid, is introduced into the dehydration tower (10) via the pipe (L11). Water is refluxed from the separation tank (12 ) to the dehydration tower (10) via the pipe (L16). This water forms an azeotrope with the azeotropic agent p-xylene, which is supplied via the pipe (L14).

An azeotrope of water and p-xylene (b.p.≈94° C.), acetic acid (b.p.≈118° C.), p-xylene (b.p.≈138° C.) and water (b.p.≈100° C.) are mixed inside the dehydration tower (10). The mixture is separated by the energy supplied through the steam heater (13).

Via the pipe (L12) at the top of the dehydration tower (10), gaseous azeotrope of water and p-xylene is discharged. And, via the pipe (L15) at the bottom of the dehydration tower (10), acetic acid, trace water and surplus p-xylene are discharged and recycled to the reactor for producing terephthalic acid (not illustrated).

Here, surplus p-xylene refers to the p-xylene which is not discharged at the top of the dehydration tower by forming the azeotrope with water but flows down to the bottom of the dehydration tower (10) when excess p-xylene has been supplied via the pipe (L10).

The azeotrope of water and p-xylene, which has been evaporated at the top of the dehydration tower (10), is condensed passing through the condenser (11) and separated at the separation tank (12). Then, water is discharged as wastewater via the pipe (L13) and the p-xylene azeotropic agent is refluxed to the dehydration tower (10) via the pipe (L14).

Also, part of the water separated at the separation tank (12) may be refluxed to the dehydration tower (10) via the pipe (L16).

If the concentration of acetic acid inside the dehydration tower (10) is too high, the efficiency of separation of acetic acid and water decreases and, thus, operation cost increases. In such a case, part of the water separated at the separation tank (12) may be refluxed to the top of the dehydration tower (10) in order to control the concentration of acetic acid inside the dehydration tower (10).

Differently from other conventional azeotropic agents, p-xylene is hardly soluble in water, with a solubility of 0.05 wt % or less, and, thus, is easily separable from water and loss of the azeotropic agent included in the wastewater can be reduced.

Also, because the p-xylene azeotropic agent is a reactant used in the production of terephthalic acid, it can be comprised with a relatively large content range. The content of p-xylene in the pipe (L15), which is discharged from the bottom of the dehydration tower (10), may be freely adjusted in the range from 0 to 65 parts by weight by controlling the amount of water refluxed to the dehydration tower (10) and the amount of p-xylene recycled to the reactor.

That is, in the 100 parts by weight of the total effluent discharged via the pipe (L15), which comprises acetic acid, water and p-xylene, the content of p-xylene is maintained within 0-65 parts by weight, preferably within 0-30 parts by weight. A p-xylene content exceeding 65 parts by weight is undesirable in view of operation load of the dehydration tower, because the amount of p-xylene may exceed that required by the reactor. However, the afore-mentioned range is not a definite one. The content needs to be determined considering the amount of p-xylene required by the reactor.

Typically, the content of water included in the 100 parts by weight of the total effluent discharged at the bottom of the dehydration tower via the pipe (L15) is 5-10 parts by weight. The larger the water content, the more water is supplied from the reactor to the dehydration tower, resulting in increase of operation load of the dehydration tower. The water content may be maintained below 5 parts by weight, but, in this case, much more energy is consumed and corrosion of the apparatus may accelerate.

Commonly used conventional azeotropic agents have several shortcomings when they are recovered to the reactor along with acetic acid. It is because the azeotropic agent, which is discharged from the bottom of the dehydration tower without forming an azeotrope with water, is lost inside the reactor by oxidation. A lot of energy is consumed to operate the dehydration tower such that the azeotropic agent is practically nonexistent at the bottom of the dehydration tower.

In contrast, because the present invention uses p-xylene, which is a reactant itself, as azeotropic agent, no further loss of the azeotropic agent occurs in the reactor. And, the content of the azeotropic agent recovered to the reactor along with acetic acid is less restricted. Besides, even in case of an accident in which the azeotropic agent is poured into the reboiler of the dehydration tower and is mixed with acetic acid, no further separation is required because the p-xylene azeotropic agent is a reactant itself.

With the conventional azeotropic agents, it is possible to increase the concentration of acetic acid. However, as the concentration of acetic acid increases, the possibility of corrosion of the material of the bottom of the dehydration tower tends increases, too. In order to prevent corrosion, costly titanium has to be used.

In addition, by increasing the amount of p-xylene discharged from the bottom of the dehydration tower (10), the content of acetic acid can be reduced relatively, thereby the corrosion problem caused by high-concentrated acetic acid can be solved. Consequently, the material used at the bottom of the dehydration tower (10) may be replaced in part from expensive titanium (Ti) to, for example, nickel alloy.

Besides, the present invention offers a further advantage that, by increasing the pressure at the top of the dehydration tower (10) above normal pressure, the temperature inside the condenser (11) increases and the condensation heat may be recovered for use.

In conventional distillation, the dehydration tower is operated such that the pressure at the top of the tower is close to normal pressure (atmospheric pressure; 1.0332 kg/cm², abs). In this case, temperature at the top of the tower becomes about 105° C. Some plants utilize this heat to produce low-pressure steam (0.75 kg/cm², abs).

However, when an azeotropic agent is used, the boiling point decreases, e.g. to about 95° C. in case of using p-xylene and to about 91° C. in case of using n-butyl acetate, and steam production becomes impossible.

By increasing the operation pressure at the top of the dehydration tower to about 1.5-1.6 kg/cm², abs, temperature at the top of the tower can be maintained at about 105-106° C., which enables production of low-pressure steam (0.75 kg/cm², abs).

### EXAMPLES

Practical and preferred embodiments of the present invention are illustrated in the following examples. However, it will be appreciated that those skilled in the art, in consideration of this disclosure, may make modifications and improvements within the spirit and scope of the present invention.

### Example 1

A mixture of water and acetic acid comprising 65 wt % of acetic acid and 35 wt % of water, which had been obtained from a reactor for producing terephthalic acid, was supplied to a dehydration tower. Construction of the dehydration tower was as follows: 20 planar distillation trays; structured packing 1,050 mm. 0.011 g/hr of p-xylene (PX) was supplied as the azeotropic agent and the dehydration tower was operated under a common operation condition (Table 1) and a special operation condition (Table 2).

The result is given in Table 3. In Table 3, the BTM (bottom) product means the product discharged from the bottom of the dehydration tower. As shown in Table 3, loss of acetic acid and p-xylene discharged as wastewater was small, which means that the cost of wastewater treatment can be saved. 9.98 kJ of energy was consumed to remove 1 g of water.

### Example 2

179 g/hr of p-xylene (PX) was supplied as the azeotropic agent and operation was performed under the condition given in Table 2. As shown in Table 3, loss of acetic acid and p-xylene discharged as wastewater was small, which means that the cost of wastewater treatment can be saved. 4.7 kJ of energy was consumed to remove 1 g of water.

### Comparative Example 1

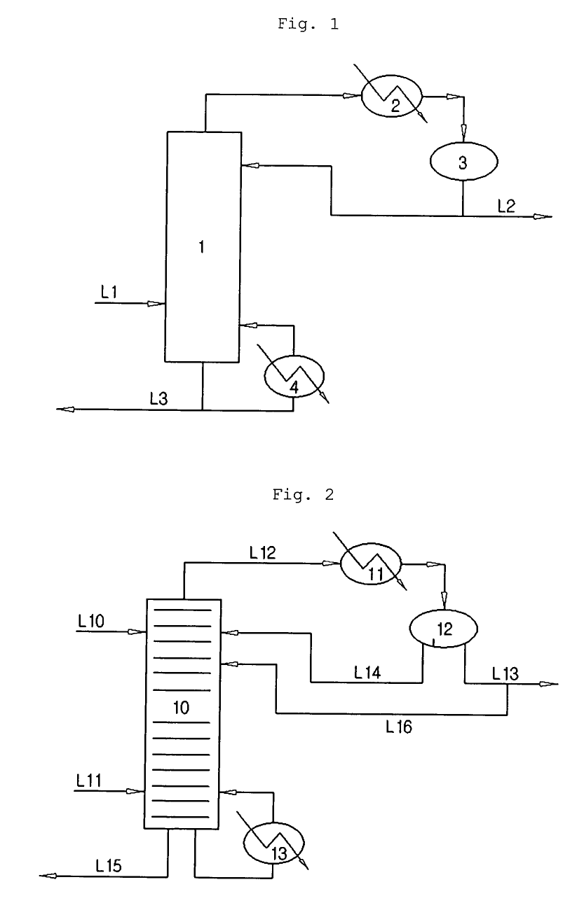

A conventional distillation apparatus illustrated in FIG. 1 was used. Operation was performed under the condition given in Table 2 without using azeotropic agent. As shown in Table 3, a lot of acetic acid was lost as wastewater, which means that the cost of wastewater treatment increases. 46.1 kJ of energy was consumed to remove 1 g of water.

TABLE 1

| | | Feed | | | | |
|---|---|---|---|---|---|---|
| | Feed | composition | | | | |
| Feed rate | temperature | (acetic | Construction of | Feed | Tray | Pressure at the top of |
| (g/hr) | (° C.) | acid/water) | dehydration tower | trays | type | dehydration tower |
| 173 | 70 | 65 wt %/35 wt % | 20 trays; | 10 trays | Sieve | Atmospheric |
| | | | structured packing | from | tray | pressure |
| | | | | bottom | | |

TABLE 2

| | Comp. | | |
|---|---|---|---|
| | Example 1 | Example 1 | Example 2 |
| Azeotropic agent | — | PX | PX |
| Azeotropic agent feed rate (g/hr) | 0 | 0.011 | 179 |
| Water evaporation rate at pipe L12 | 610 | 140 | 60 |
| (g/hr) | | | |
| Azeotropic agent evaporation rate at | 0 | 282 | 117 |
| pipe L12 (g/hr) | | | |
| Water + azeotropic agent reflux rate | 580 | 373 | 306 |
| (g/hr) | | | |
| Wastewater discharge rate (g/hr) | 30 | 49 | 49 |
| Heating rate (kJ/hr) | 1383 | 489 | 232 |
| Energy consumption per 1 g of | 46.1 | 9.98 | 4.7 |
| wastewater (kJ/g) | | | |

TABLE 3

| | Comp. Example 1 | Example 1 | Example 2 |
|---|---|---|---|
| Wastewater composition, wt % (PX/acetic | 0/2.8/97.2 | 0.01/0.29/99.7 | 0.01/0.001/99.98 |
| acid/water) | | | |
| BTM composition, wt % (PX/acetic | 0/78/22 | 0.003/90.5/9.5 | 59.2/37.1/3.7 |
| acid/water) | | | |
| Temperature at the top of the dehydration | 100 | 95.3 | 94.9 |
| tower (° C.) | | | |
| Temperature at the bottom of the | 105 | 109 | 107 |
| dehydration tower (° C.) | | | |

As shown in Table 2, outstandingly less heat is required to remove a given amount of water in the proposed azeotropic distillation process compared to that in the conventional distillation process. Thus, it was confirmed that energy consumption can be greatly reduced by using p-xylene as an azeotropic agent.

Moreover, if the concentration of the azeotropic agent at the bottom of the dehydration tower is maintained high, as shown in Example 2, it is possible to replace the material of the bottom of the dehydration tower in part from expensive titanium to nickel alloy.

### INDUSTRIAL APPLICABILITY

As apparent from the above description, the recovering method of acetic acid in accordance with the present invention provides the following advantages by using p-xylene as an azeotropic agent.

First, while using conventional azeotropic agents is restricted a lot in the concentration range of azeotropic agent in the recovered acetic acid, the present invention is advantageous in that. It is not restricted in the content of azeotropic agent in the recovered acetic acid because the azeotropic agent, p-xylene, itself is a reactant.

Second, even in case of an accident in which the azeotropic agent is poured into the reboiler of the dehydration tower and is mixed with acetic acid, no further separation is required because the p-xylene azeotropic agent is a reactant itself.

Third, by increasing the concentration of the p-xylene azeotropic agent discharged from the bottom of the dehydration tower, it is possible to replace the material of the bottom of the dehydration tower in part from expensive titanium to nickel alloy and, thus, construction cost can be saved.

Fourth, because less p-xylene is included in the water layer separated at the top of the dehydration tower, loss of the azeotropic agent is reduced and the cost of wastewater treatment can be saved.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent modifications do not depart from the spirit and scope of the present invention as set forth in the appended claims.

1. A recovering method of acetic acid from effluent from a terephthalic acid production process using an apparatus comprising:
a dehydration tower for recovering acetic acid from a mixture of water and acetic acid, which is produced from catalytic oxidation of p-xylene with air in the presence of acetic acid, by azeotropic distillation using an azeotropic agent;
a condenser for condensing gaseous azeotropic agent and water discharged from the top of the dehydration tower; and
a separation tank for removing water by separating the azeotropic agent from water,
wherein p-xylene is used as the azeotropic agent to form an azeotrope with water, which is discharged from the top of the dehydration tower, and remaining p-xylene is discharged from the bottom of the dehydration tower along with acetic acid.

2. The recovering method of acetic acid as set forth in claim 1, wherein the p-xylene is comprised in the gaseous azeotrope discharged from the top of the dehydration tower within 1.95-2.2 parts by weight per 1 part by weight of water.

3. The recovering method of acetic acid as set forth in claim 1, wherein the acetic acid including p-xylene, which is discharged from the bottom of the dehydration tower, is recycled to a reactor for the production of terephthalic acid.

4. The recovering method of acetic acid as set forth in claim 3, wherein the p-xylene is comprised in the acetic acid within 0-65 parts by weight per 100 parts by weight of the total effluent discharged from the bottom of the dehydration tower.

5. The recovering method of acetic acid as set forth in claim 3, wherein part of the water separated at the separation tank is refluxed to the top of the dehydration tower.

6. The recovering method of acetic acid as set forth in claim 3, wherein the top of the dehydration tower is operated at a pressure above normal pressure in order to recover energy from the condenser.

7. The recovering method of acetic acid as set forth in claim 1, wherein the azeotropic agent further comprises, in addition to p-xylene, a material selected from the group consisting methyl acetate, isobutyl acetate, n-butyl acetate and butanol.

8. The recovering method of acetic acid as set forth in claim 7, wherein the p-xylene is comprised within 50-100 wt % per 100 wt % of the total azeotropic agent.

9. The recovering method of acetic acid as set forth in claim 2, wherein the acetic acid including p-xylene, which is discharged from the bottom of the dehydration tower, is recycled to a reactor for the production of terephthalic acid.
